(19) United States
(12) Patent Application Publication
(10) Pub. No.: US 2025/0339313 A1
(43) Pub. Date: Nov. 6, 2025

(54) VENTED MULTI-DOSE OCULAR FLUID DELIVERY SYSTEM

(71) Applicant: Bausch + Lomb Ireland Limited, Dublin (IE)

(72) Inventors: Yehuda IVRI, Newport Coast, CA (US); Reynaldo QUINTANA, Menlo Park, CA (US); Daniel V. PALANKER, Sunnyvale, CA (US)

(21) Appl. No.: 18/862,267

(22) PCT Filed: May 1, 2023
(86) PCT No.: PCT/US2023/066423

§ 371 Date: Nov. 1, 2024

Related U.S. Application Data
(60) Provisional application No. 63/337,372, filed on May 2, 2022.

(51) Int. Cl. A61F 9/00; A61M 35/00; A61M 39/24

(52) U.S. Cl. CPC A61F 9/0008; A61M 35/003; A61M 39/24; A61M 2202/0007; A61M 2202/04; A61M 2205/3331; A61M 2205/7545; A61M 2210/0612

(57) ABSTRACT

Handheld dispensing system devices are disclosed. Aspects of the dispensing devices include a first pathway for directing the liquid from an ampoule to a chamber having an aperture through which the liquid from the ampoule can be dispensed. The device includes an actuator to oscillate a membrane to dispense the liquid through the membrane. The device further includes a second pathway in fluid communication with the interior of the ampoule and the atmosphere to equalize the pressure in the ampoule as liquid is dispensed. The second pathway can be sufficiently designed to prevent liquid from escaping through the second pathway.

### CROSS-REFERENCE TO RELATED APPLICATION(S)

This application claims priority to, and the benefit of, U.S. Provisional Application No. 63/337,372, filed May 2, 2022, for all subject matter common to both applications. The disclosure of the provisional application is hereby incorporated by reference in its entirety.

### FIELD OF THE INVENTION

This disclosure relates to topical ocular delivery of ophthalmic medications.

### BACKGROUND

Currently, pharmaceutical fluids are typically delivered to the eye surface using an eye-drop bottle. This method has multiple drawbacks: (1) Patients cannot aim well and often miss the eye; (2) Volume of a drop from a bottle is not well-defined and is too large (on the order of 50 μL) for the tear film on the cornea to absorb it—the tear film can hold no more than about 7 μL; (3) Very often patients blink during the drop delivery, so that part of the drop lands on the eyelid, and the rest is wiped off the cornea.

### SUMMARY

The instant disclosure provides for methods and a device that addresses these problems by (1) delivering a substantially precise amount of fluid or liquid; (2) delivering a micro-dose that the tear film can hold (e.g., <10 uL); (3) delivering the dose of liquid within the blink reflex time (e.g., about 100 ms), and (4) using a visual aiming structure for precise self-administration of the liquid.

In an embodiment, a handheld device for dispensing a liquid to an eye of a patient is provided. The device includes a first pathway for directing the liquid from an ampoule to a chamber having a) an aperture through which the liquid from the ampoule can be dispensed, and b) a membrane designed to hydrodynamically excite and dispense the liquid through the aperture. The device also includes an actuator designed to oscillate a rod that engages the membrane so that the membrane can hydrodynamically excite the liquid to open the aperture and dispense the liquid therethrough. The device further includes a second pathway having a first end in fluid communication with at least one of the first pathway and the ampoule, a second end exposed to atmosphere, and a stop designed to prevent the liquid from escaping through the second end of the second pathway.

In some embodiments, the chamber can further include a valve member extending from the membrane, where the valve member is engaged within the aperture in a closed configuration and disengaged from the aperture when the actuator oscillates the connecting rod. In an embodiment, the valve member and the membrane can be a unitary part.

In some embodiments, the device can further include a cap designed to cover the aperture, where the cap can include a plug that is designed to enter the aperture to displace the liquid in the aperture.

In some embodiments, the second pathway can further include an air filter designed to sterilize air flowing from the atmosphere into the ampoule as the liquid is dispensed. In an embodiment, the air filter can be designed to remove particles larger than 0.22 microns. In some embodiments, the air filter can be designed to remove contaminants and microorganisms from air entering the ampoule. In an embodiment, the air filter can be a hydrophobic material such that air filtration and air flow rate into the ampoule are minimally affected by contact between the air filter and the liquid within the ampoule.

In some embodiments, the stop can be a one-way valve designed to prevent the liquid in the ampoule from exiting the pathway. In an embodiment the second pathway can be hermetically sealed. In some embodiments, the second pathway can further be designed to equalize pressure.

In another embodiment, a handheld device for dispensing a liquid to a site of interest is provided. The device includes a first pathway for directing the liquid from an ampoule to a chamber having a) a membrane and pin designed to hydrodynamically excite and dispense the liquid and b) a sealable aperture through which the liquid from the ampoule can be dispensed, where the pin is received within the aperture to seal the aperture. The device also includes an actuator designed to oscillate the membrane and the pin to disengage the pin from the aperture and dispense the liquid therethrough. The device further includes a second pathway having a first end in fluid communication with at least one of the first pathway and the ampoule, a second end exposed to atmosphere, and a stop designed to prevent the liquid from escaping through the second end of the second pathway, regardless of orientation of the liquid within the ampoule.

In some embodiments, the device further can include a cap designed to cover the aperture, where the cap can include a plug that is designed to enter the aperture to displace the liquid in the aperture.

In some embodiments, the second pathway can include an air filter designed to sterilize air flowing from the atmosphere into the ampoule as the liquid is dispensed. In an embodiment, the air filter can be designed to remove particles larger than 0.22 microns. In some embodiments, the air filter can be designed to remove contaminants and microorganisms from the air entering the ampoule. In an embodiment, the air filter can be a hydrophobic material such that air filtration and air flow rate into the ampoule are minimally affected by contact between the air filter and the liquid within the ampoule.

In some embodiments, the stop can be a one-way valve designed to prevent the liquid in the ampoule from exiting the second pathway. In an embodiment, the second pathway can be hermetically sealed. In some embodiments, the second pathway can be further designed to equalize pressure.

### BRIEF DESCRIPTION OF DRAWINGS

These and other characteristics of the present disclosure will be more fully understood by reference to the following detailed description in conjunction with the attached drawings, in which:

FIG. 1 shows geometry for delivering liquid to an eye of a patient according to an embodiment.

FIG. 2 is an exterior view, according to an embodiment.

FIG. 3A is a cross sectional view, according to an embodiment.

FIGS. 3B-C show operation of the embodiment of FIG. 3A according to an embodiment.

FIGS. 4A-B show an example of venting according to an embodiment.

FIG. 5 is a cross sectional view, according to an embodiment.

FIG. 6 shows a conical spring according to an embodiment.

FIG. 7 shows an embodiment having anti-clog features, according to an embodiment.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure generally relates to handheld devices for dispensing liquids to a desired location or site of interest, e.g., an eye of a patient. For example, the present disclosure relates to a device with an ampoule for containing liquid, an assembly with a membrane, which when the membrane can be acted upon, can create hydrodynamic pressure in the liquid towards an aperture being sealed by a member. An actuator can be provided to oscillate the sealing member connected to the membrane to create the hydrodynamic excitation in the liquid while unsealing the aperture through the oscillations provided to the sealing member such that liquid can be dispensed through the aperture.

With respect to FIGS. 1-7, wherein like parts are designated by like reference numerals throughout, illustrate an example embodiment or embodiments of the device of dispensing liquid to an eye, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

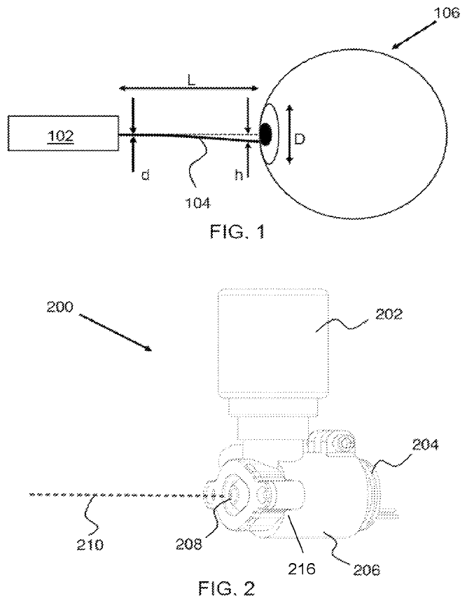

FIG. 2 shows a perspective view of a liquid ejection unit according to an embodiment. The liquid ejection unit 200 can be suitable for use in delivering liquid, such as preservative-free pharmaceutical liquid, to the surface of the eye. Alternatively, the liquid ejection unit 200 can be used with any desired liquid. The liquid ejection unit 200, in one embodiment, can comprise a thermoplastic body 206 formed with a liquid chamber 216 and connected to a liquid supply ampoule 202. In an embodiment, the ejection unit 200, as illustrated in FIG. 2, can also include a nozzle, or aperture 208 through which liquid stream 210 can be dispensed.

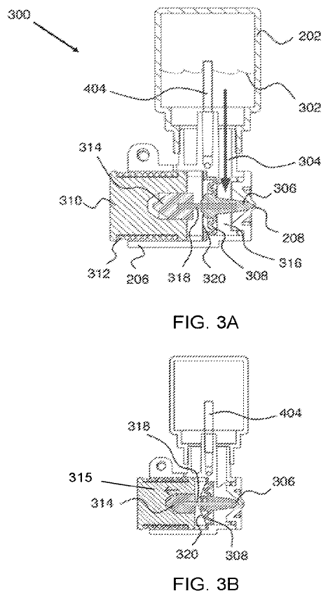

FIG. 3A illustrates a cross-sectional view of a liquid ejection device 300 according to an embodiment. Similar to the liquid ejection unit 200, the liquid ejection device 300, in an embodiment, can include a thermoplastic body 206 defining a chamber 316 connected to the liquid supply ampoule 202 containing a liquid 302. The liquid ejection device 300 can also include, in an embodiment, an aperture 208 through which the liquid 302 can be ejected. The device 300 can, in an embodiment, further include a membrane 308 arranged opposite the chamber 316 from the aperture 208. Membrane 308, in an embodiment, can include an integral needle 306, or valve member, such that they can be one component or a single unitary part. Alternatively, the needle 306 may not be integral with the membrane 308. In some embodiments, the needle 306 and the membrane 308 can be connected to an electromagnetic transducer 310 via a link member 318. In some embodiments, the electromagnetic transducer 310 can be any suitable actuator, e.g., a coin cell motor. In an embodiment, upon application of an electrical pulse to the electromagnetic transducer 310, electric current can flow through the coil 312 and a magnetic force can be developed which can pull plunger 314 backward against a spring 320. FIG. 3A, shows a direction 304 of liquid 302 flow from ampoule 202 to chamber 316.

FIG. 3B shows the device of FIG. 3A following an application of an electrical pulse to electromagnetic transducer 310, according to an embodiment. As a result of the magnetic force, the plunger 314 can be actuated such that the plunger 314 can be pulled by the electromagnetic transducer 310 in a direction indicated by the arrow 315. Membrane 308 can be connected to plunger 314 by linkage member 318 and can also pulled back. FIG. 3C shows the situation when the electromagnetic transducer 310 can be de-energized. In the illustrated embodiment, the spring 320 pushes the membrane 308 back to its original position, so the aperture 208 can be closed and the chamber can be hermetically sealed and to prevent microbial ingress. This actuation can be repeated in quick succession a multitude of times to eject liquid 302 from the device 300.

For example, in an embodiment, the electromagnetic transducer 310 can be energized with a pulsatile or alternating (AC) current, such that membrane 308 can be oscillated to generate pressure in the liquid 302. The pressure in the liquid 302 then can result in a stream 210 being ejected from the aperture 208. In an embodiment, the operating frequency can be from about 10 to about 500 Hz and more specifically from about 50 to about 200 Hz. In an embodiment, the membrane 308 can be made of silicon having a hardness durometer between about 50 to about 70 (shore A), and the displacement of plunger 314 can be about 200 μm. In some embodiments, the flow of liquid 302 can be produced only in the outward direction, as shown by stream 210, such that the liquid 302 flow can prevent microbial ingress even when the aperture 208 may be open.

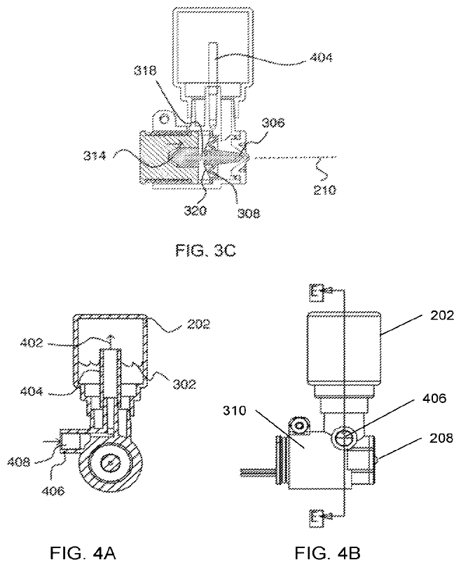

In some embodiments, as shown in FIGS. 4A-4B, the liquid ejection device 300 can include a venting arrangement to equalize the pressure inside the ampoule 202 with the ambient atmospheric pressure. Here, FIG. 4A illustrates a cross sectional view along line E-E of FIG. 4B. In an embodiment, the venting system can include an air inlet vent tube 404 that can extend beyond the level of liquid 302 in the ampoule 202. It should be noted that the vent tube 404 may be placed above the liquid 302 level in any orientation of the device 300 of FIGS. 4A-B. In the illustrated embodiment, vent tube 404 can be connected to venting outlet 406, which can be open to the atmosphere. Vent tube 404, being in fluid communication with the atmosphere and the ampoule 202, can equalize the pressure within the ampoule 202, to prevent a pressure vacuum from being created as the liquid 302 can be dispensed through aperture 208. In an embodiment, a filter 408 can be placed in the venting outlet 406 such that the vented air flowing to the ampoule 202 can be filtered to prevent penetration of potential airborne contamination, such as microbes. In an embodiment, filter 408 can filter away particles with a size greater than 1 μm and more preferably greater than 0.5 μm, still more preferably greater than 0.2 μm. In this way, the system can be isolated from microbial contamination, even though air 402 can enter the ampoule 202 as liquid can be emitted.

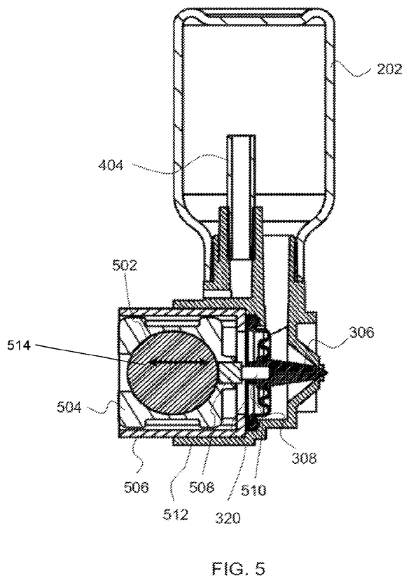

In the preceding examples, diaphragm or membrane 308 can be driven with a solenoid. In alternative embodiments, as illustrated in FIG. 5, the membrane 308 can be driven by using a coin vibration motor 502. More specifically, needle 306 can engage a membrane 308. The needle 306 and membrane 308 assembly can, in an embodiment, be over molded with a magnetic steel pin 510. In one embodiment, coin vibrator motor 502 can be JINLONG MACHINERY & ELECTRONICS CO., LTD. model #C1026B002F. In an embodiment, motor holder 504 can be a plastic molded component which holds the motor 502 such that it can slide along rails. Rail guide component 506 can, in an embodiment, be a plastic molded component that can provide the above-mentioned rail guides for motor holder 504 to slide within. In some embodiments, magnetic steel pin 510 can be molded into the membrane 308 and needle 306 assembly. Housing 512 can, in an embodiment, hold all the components together.

In one embodiment, an electromagnetic transducer 310 can be attached to the housing 512, and when energized, can pull the membrane 308 rearward against a spring 320 in the chamber 316. When the electromagnetic transducer 310 can, in an embodiment, be turned off, the spring 320 returns the membrane 308 to its original position with the aperture 208 closed. When the electromagnetic transducer 310 can, in an embodiment, be energized with pulsatile or alternating current, the membrane 308 can be consequently oscillated, which in turn generates pressure in the liquid 302. At the correct frequencies, the pressure can be sufficient to eject a stream of liquid 302 from the aperture 208. In this embodiment, a typical range of frequencies can be in a range of about 10 Hz to about 500 Hz, more optimally about 50 to about 200 Hz. The diameter of the aperture 208, velocity of the liquid 302 ejection, and duration of the electromagnetic burst, in an embodiment, can be optimized to deliver the required amount of liquid 302 within the required amount of time. In accordance with one embodiment of the invention, the actuation pulse duration can be about 250 ms or less, and in some embodiments it can be about 100 ms or less. It should be appreciated that ‘actuation pulse duration’ refers to the length of time the electromagnetic transducer may be energized so as to pull the needle 306 out of the aperture 208 in a single actuation pulse.

In an alternative embodiment, the actuator can be a coin vibration motor 502 which can have an eccentric weight off its axis of rotation (the axis of rotation can be perpendicular to the plane of FIG. 5). Because the weight can be off axis, as the motor rotates, the unbalanced weight causes the motor 502 to vibrate primarily in the plane of FIG. 5. By placing the coin vibration motor 502 in a plastic motor holder 504, which fits into corresponding rails (in rail guide component 506), the coin vibration motor 502 can be constrained, in an embodiment, so it can only move linearly, as shown by arrows 514 (e.g., left to right on FIG. 5). As a result of this physical constraint, when the motor 502 can rotate, in the illustrated embodiment, it can be only allowed to oscillate left to right, rather than to vibrate in a plane. The coin vibration motor 502 can be coupled to the membrane 308, consequently as the motor 502 oscillates left to right, the membrane 308 may also be vibrated left to right. Again, in the illustrated embodiment, the ejected liquid stream 210 can be generated in the same manner as described above—i.e., the needle 306 moves back and forth in the aperture 208 to eject the liquid 302. In some embodiments, the tip of needle 306 and/or the aperture 208 it engages with can include or be made of an anti-microbial material.

To aid in sealing aperture 208 with needle 306, the embodiment of FIG. 5 can include a disk-shaped spring 320. The spring 320 can be slightly deformed out of plane during assembly which serves to transmit force to needle 306. This force or load can keep the needle 306 pressed up against the orifice or aperture 208 to close the flow path. In the illustrated embodiment, without the spring 320, the force required to push the needle 306 and open aperture 208 can be very low which can result in liquid 302 leaking. Additionally, the spring 320 has a spring constant which can be important for ensuring the correct frequency and amplitude of oscillation of the needle 306 when the motor 502 can be energized. Also important to the illustrated embodiment can be that without the spring 320 keeping the aperture 208 in a closed and sealed position with needle 306, the only force maintaining the needle 306 and aperture 208 in a closed position can be the stiffness of membrane 308. The membrane 308 can, in an embodiment, be made of an elastomer. For most elastomers, mechanical properties vary significantly even with modest temperature changes. In an embodiment with the spring 320, a significant portion of the load applied to the needle 306 comes from the spring 320, not the membrane 308. Because the mechanical properties of spring steel (e.g., the material of the spring 320) can be more consistent for the same temperature change, adding the spring to the illustrated embodiment can make the system performance more consistent.

In an alternative embodiment, the coin vibration motor 502 can be coupled to the diaphragm or membrane 308 via an optional magnet 508. Magnet 508 can be fixed to motor holder 504 which can be affixed to the coin vibration motor 502. When the magnet 508 may be close to the magnetic steel pin 510, in this embodiment, the two latch can together and the motor 502 can be thereby coupled to the membrane 308. This can be an advantageous assembly feature for an embodiment, because the motor 502 can be easily added to the system without the need for tight tolerances and the motor 502 can be added at several different steps of the assembly process, depending on the manufacturing requirements.

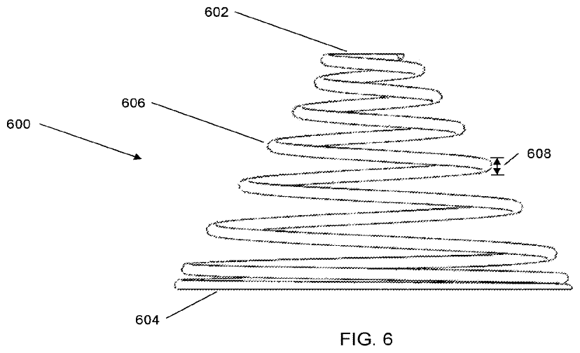

In an alternative embodiment, the disc spring 320 of FIG. 5 can be replaced with a conical spring 600, as shown in FIG. 6. In the illustrated embodiment, a conical spring 600 can be similar to a conventional compression spring made of wire, but instead of being wound with a constant diameter, the diameter can begin at a large diameter 604 and can get progressively smaller to small diameter 602, so that the spring has the shape of a cone. In an embodiment, when the conical spring 600 can be fully compressed, the coils 606 can nest within each other so the spring 600 can become flat, only being as thick as the diameter of the wire 608 it can be wound from. Thus, a fully compressed conical spring 600 can fit in a similar form factor as the disc spring 320 of FIG. 5 and can serve the same function. The conical spring 600 can be cheaper and can be easier to get a wide range of spring constants and operating deflections compared to the disc spring 320.

For convenient aiming of the liquid dispenser device 102, the device 102 can be placed close to the eye, but not touching the eyelashes or eyebrow of the user. Therefore, with reference to FIG. 1, the device 102 can be in the range of approximately L=1-10 cm from the eye, or more optimally about 2 to 6 cm. The cornea can be about D=12 mm in diameter, i.e., about 6 mm in radius. To ensure that the liquid 104 can be delivered approximately to the middle of the cornea, the jet of emitted liquid 104 may not deflect under gravity by more than about half the cornea radius, i.e., no more than about h=3 mm. As shown in FIG. 1, vertical deflection h of the projectile ejected horizontally with velocity v over a distance L is: h=g*L/(2v²), where g is gravity. To ensure that vertical deflection does not exceed h, horizontal jet velocity may exceed v=L*(g/2 h)^{0.5}. For example, where L=5 cm, g=9.8 m/s², h=3 mm, we obtain v=2 m/s. For L=5 cm and h=1 mm, velocity should be about v=3.6 m/s, and for L=10 cm, h=1 mm, velocity v=7.2 m/s. Therefore overall, jet velocity can be in the range of about 1-10 m/s, and more optimally about 2-4 m/s. Velocities higher than those may cause discomfort to the patient and can damage to the cornea.

The stream of liquid 104 can reach the eye within a few milliseconds from the moment of dispensing (t=L/v, in the range of about 1-100 ms). As soon as the liquid 104 can touch the cornea or eye 106, it can trigger the blink reflex, which can typically take about T=100 ms. To prevent the drug from being blocked by the eyelid, the liquid 104 can be delivered before the eye 106 closure. For the required volume V to be delivered within the time T with the jet velocity v, the jet cross-sectional area can be S=V/(T*v). Since, for a round aperture 208, S=π*d²/4, its diameter d=(4V/(πT*v))^{0.5}. For example, for v=2 m/s, T=100 ms, V=10 μL, we obtain d=250 μm. For v=1 m/s, d=350 μm, and for v=7 m/s, d=130 μm. Therefore, the aperture diameter of the device 102 can be in the range of approximately 200-600 μm, and more optimally about 400-550 μm. Alternatively, several apertures 208 could be used to produce several parallel streams for faster delivery.

Another attribute of an embodiment of the system can be the prevention of microbial ingress to the contained liquid 302 during storage or use. As with any closed system, as liquid 302 can be ejected, air should be introduced to replace the ejected volume and thereby balance the pressure (venting) within the ampoule 202. To preclude microbial ingress, the air can be, in an embodiment, be introduced via a special inlet preferably having a 0.2 μm filter. Ideally, the device should operate such that liquid 302 can be ejected through the aperture any time it can be opened, thereby preventing the air ingress through it.

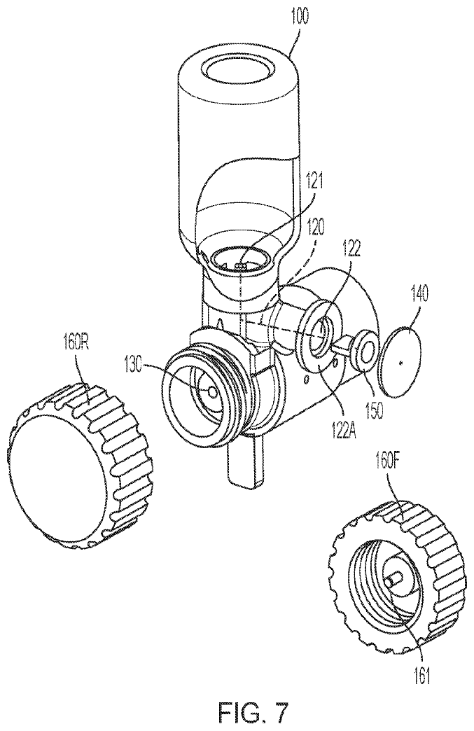

Now in reference to FIG. 7, and to achieve this pressure balance, an alternative venting system can be provided. For the sake of brevity, like parts described in the earlier embodiments will not be described. The alternative venting system embodiment illustrated in FIG. 7 can provide sterile venting of ambient air into the ampoule 202 by providing a pathway or venting channel 120, which can be in fluid communication with the interior of the ampoule 202 and the atmosphere. The venting channel 120 can have an air outlet port 121 inside the ampoule 202 and an external air inlet port 122 that can be open to the atmosphere. In the illustrated embodiment, the route of the venting channel 120 can be illustrated by the broken line. In alternative embodiments, the venting channel 120 can be located in different locations, such as on the side, top, or bottom of ampoule 202. Further, while illustrated as a curved line, venting channel 120 can, in alternative embodiments, extend along a path of any shape. For example, venting channel 120 can be a straight line. The venting channel 120 can, in an embodiment, equalize the pressure between the ampoule 202 and the atmospheric pressure. In the previous embodiments, a venting tube 404 may be disclosed and can result in a similar function. The embodiment shown in FIG. 7 has the added benefit of not requiring a venting tube, which would require the top of the tube 404 to be disposed above of the liquid 302 and, further, removal of the tube 404 can provide more volume for liquid 302 inside ampoule 202.

In some embodiments, there can be a stop, or valve 150, in the venting channel 120 which can permit the flow of air into the ampoule 202 as liquid 302 can be dispensed through aperture 208 while preventing liquid 302 from escaping, regardless of orientation of the device 100. In one embodiment, the stop in the venting channel 120 can include a one-way valve 150, or check valve, positioned between the filtration member 140 and the external air inlet port 122. In an embodiment, the valve 150 can be a duckbill valve model DU02.001SD.v1 made by MiniValve. In alternative embodiments, the check valve can be a ball check valve, a diaphragm check valve, a stop-check valve, or other like one way valves. When assembled, valve 150 can, in an embodiment, be disposed in the venting channel 120 and filtration member 140 can be welded, or otherwise secured, to surface 122A, thereby hermetically sealing off the venting channel 120 at the external air inlet port 122. Although welding filtration member 140 to surface 122A can be one method for hermetically sealing off the venting channel 120, in an alternative embodiment, the one-way valve 150 can create an hermetic seal itself, without welding the surfaces together. Any hermetically sealing structure or valve 150 can be utilized. In other words, any one-way valve or check valve that will permit the flow of air through the valve while preventing the release of liquid through the valve can be utilized.

The venting system can, in an embodiment, further include a filtration member 140 capable of removing particles larger than about 0.22 μm. The filtration member can be made of hydrophobic material such that air filtration and flow rate can be minimally affected by the contact of the filtration member 140 with aqueous solution. In that embodiment, the resistance to air flow may not be affected by contact with the liquid 302 or aqueous solution with the hydrophobic 0.22 micron filter. The hydrophobic filter can provide sterility assurance, high flow rates and high throughput. In an embodiment, the filtration member can be made of polyvinylidene fluoride (PVDF) that can reliably eliminate contaminants and microorganisms. Alternatively, the filtration member can be made out of metal, ceramic, or another plastic so long as the filtration member 140 can effectively sterilize the air being introduced into ampoule 202 through venting channel 120. In some embodiments, the venting channel 120 can position the one-way valve 150 between the filtration member 140 and the external air inlet port 122 such valve 150 may be used to eliminate physical contact of liquid 302 with the filtration member.

Still in reference to FIG. 7, the illustrated embodiment of a dispensing device 100 can further include a means to prevent clogging of the dispensing aperture 130. This arrangement can be particularly effective when the liquid 302 can leave solid residue in the aperture 130. The anti-clog system can include, in an embodiment, an elongated dispensing port, or aperture 130, through which droplets can be dispensed and further includes a screw-on cap with concentric pin 161, or plug. The screw on cap can be shown in rear and frontal view 160R and 160F, respectively. When the cap can, in an embodiment, be engaged with the dispensing system, pin 161 can enter the dispensing aperture 130 such that residual liquid 302 in the dispensing port can be displaced out. In one embodiment, the pin 161 and the aperture 130 can be cylindrical or slightly tapered. In alternative embodiments, the pin 161 and aperture 130 can be any complimentary surfaces such that the pin 161 displaces residual liquid 302 from the aperture 130. In alternative embodiments, aperture 130 can be flat, geometrical, or any shape that facilitates liquid 302 dispensing therethrough.

In one method of use, an embodiment of the device 100 can be activated to actuate the actuator or transducer 310, which can cause oscillations of the membrane 308 and needle 306. These oscillations to the membrane 308 and needle 306 can hydrodynamically excite the liquid 302 within chamber 316 and open aperture 130, permitting the device 100 to dispense liquid 302 through aperture 130. As liquid 302 can be dispensed, air flows through the venting channel 120 and into the ampoule 202. In this way, the pressure inside the ampoule 202 can equalize to the atmospheric pressure while the device can be dispensing liquid 302, preventing a pressure vacuum from occurring. Once dispensed, or while dispensing, liquid 302 can flow from ampoule 202 into chamber 316, replacing the dispensed liquid 302 such that the process can continue and repeat as described.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that can be “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

What is claimed is:

1. A handheld device for dispensing a liquid to an eye of a patient, the device comprising:
a first pathway for directing the liquid from an ampoule to a chamber having a) an aperture through which the liquid from the ampoule can be dispensed, and b) a membrane designed to hydrodynamically excite and dispense the liquid through the aperture;
an actuator designed to oscillate a rod engaging the membrane so that the membrane can hydrodynamically excite the liquid to open the aperture and dispense the liquid therethrough; and
a second pathway having a first end in fluid communication with at least one of the first pathway and the ampoule, a second end exposed to atmosphere, and a stop designed to prevent the liquid from escaping through the second end of the second pathway.

2. The device of claim 1, the chamber further comprising:
a valve member extending from the membrane, wherein the valve member is engaged within the aperture in a closed configuration and disengaged from the aperture when the actuator oscillates the rod.

3. The device of claim 2, wherein the valve member and the membrane are a unitary part.

4. The device of claim 1, the device further comprising:
a cap designed to cover the aperture, where the cap includes a plug that is designed to enter the aperture to displace the liquid in the aperture.

5. The device of claim 1, the second pathway further comprising:
an air filter designed to sterilize air flowing from the atmosphere into the ampoule as the liquid is dispensed.

6. The device of claim 5, wherein the air filter is designed to remove particles larger than 0.22 microns.

7. The device of claim 5, wherein the air filter is designed to remove contaminants and microorganisms from air entering the ampoule.

8. The device of claim 5, wherein the air filter is a hydrophobic material such that air filtration and air flow rate into the ampoule are minimally affected by contact between the air filter and the liquid within the ampoule.

9. The device of claim 1, wherein the stop is a one-way valve designed to prevent the liquid in the ampoule from exiting the pathway.

10. The device of claim 1, wherein the second pathway is hermetically sealed.

11. The device of claim 1, wherein the second pathway is designed to equalize pressure.

12. A handheld device for dispensing a liquid to a site of interest, the device comprising:
a first pathway for directing the liquid from an ampoule to a chamber having a) a membrane and pin designed to hydrodynamically excite and dispense the liquid and b) a sealable aperture through which the liquid from the ampoule can be dispensed, where the pin is received within the aperture to seal the aperture;
an actuator designed to oscillate the membrane and the pin to disengage the pin from the aperture and dispense the liquid therethrough; and
a second pathway having a first end in fluid communication with at least one of the first pathway and the ampoule, a second end exposed to atmosphere, and a stop designed to prevent the liquid from escaping through the second end of the second pathway, regardless of orientation of the liquid within the ampoule.

13. The device of claim 12, the device further comprising:
a cap designed to cover the aperture, where the cap includes a plug that is designed to enter the aperture to displace the liquid in the aperture.

14. The device of claim 12, the second pathway further comprising:
an air filter designed to sterilize air flowing from the atmosphere into the ampoule as the liquid is dispensed.

15. The device of claim 14, wherein the air filter is designed to remove particles larger than 0.22 microns.

16. The device of claim 14, wherein the air filter is designed to remove contaminants and microorganisms from the air entering the ampoule.

17. The device of claim 14, wherein the air filter is a hydrophobic material such that air filtration and air flow rate into the ampoule are minimally affected by contact between the air filter and the liquid within the ampoule.

18. The device of claim 12, wherein the stop is a one-way valve designed to prevent the liquid in the ampoule from exiting the second pathway.

19. The device of claim 12, wherein the second pathway is hermetically sealed.

20. The device of claim 12, wherein the second pathway is designed to equalize pressure.
